(19) United States
(12) Patent Application Publication
(10) Pub. No.: US 2024/0207573 A1
(43) Pub. Date: Jun. 27, 2024

(54) ULTRASOUND-PLACED PAIN MANAGEMENT SYSTEM AND METHOD WITH SUBCUTANEOUS CATHETER

(71) Applicant: Maho Med Tech, LLC, Charlotte Amalie, VI (US)

(72) Inventors: Shawn D Schumacher, Corvallis, OR (US); John M Matheson, St. Thomas, VI (US)

(21) Appl. No.: 18/601,021

(22) Filed: Mar. 11, 2024

Related U.S. Application Data
(63) Continuation-in-part of application No. 18/382,327, filed on Oct. 20, 2023.
Application No. 18/382,327 is a continuation-in-part of application No. 18/102,231, filed on Jan. 27, 2023.
Application No. 18/102,231 is a continuation-in-part of application No. 17/939,631, filed on Sep. 7, 2022, now Pat. No. 11,654,260.
Application No. 17/939,631 is a continuation-in-part of application No. 17/518,815, filed on Nov. 4, 2021, now abandoned.
(60) Provisional application No. 63/418,866, filed on Oct. 24, 2022.
(60) Provisional application No. 63/200,204, filed on Feb. 21, 2021.

(51) Int. Cl. A61M 25/01; A61M 5/172; A61M 25/00; A61N 1/05; A61N 1/36

(52) U.S. Cl. CPC A61M 25/0108; A61M 5/172; A61M 25/0068; A61N 1/0504; A61N 1/36071

(57) ABSTRACT

A pain management system includes a medication dispenser located external to a patient. A subcutaneous port is placed internally to the patient and receives a quantity of medication. The port can be filled from a syringe or medication dispensing system. An echogenic catheter can be placed in proximity to a patient's nerve or nerve center using a point-of-care ultrasound imaging system. A pain management method includes the steps of placing a subcutaneous port using ultrasound imaging for guidance and administering pharmacologic agents via a catheter connected to the port. Alternative embodiment catheter systems include echogenic tips, a stylet, and multiple internal coil sections to facilitate accurate placement. Alternative embodiment pain management methods include the steps of utilizing such features for optimizing patient outcomes.

### CROSS-REFERENCE TO RELATED APPLICATIONS

This application is a continuation-in-part of and claims priority in U.S. patent application Ser. No. 18/382,327, filed Oct. 20, 2023, which is a continuation-in-part of and claims priority in U.S. patent application Ser. No. 18/102,231, filed Jan. 27, 2023 which is a continuation-in-part of and claims priority in U.S. patent application Ser. No. 17/939,631 filed Sep. 7, 2022, now U.S. Pat. No. 11,654,260, Issued May 23, 2023, which is a continuation-in-part of and claims priority in U.S. patent application Ser. No. 17/17/518,815, Filed Nov. 2, 2021 which claims priority in U.S. Provisional Patent Application Ser. No. 63/200,204, filed Feb. 21, 2021, and also claims priority in U.S. Provisional Patent Application Ser. No. 63/418,866, filed Oct. 24, 2022, all of which are incorporated herein by reference in their entireties.

### BACKGROUND OF THE INVENTION

### 1. Field of the Invention

The present invention relates generally to catheters, and in particular to a catheter system with an implantable port for administering anesthetics, e.g., nerve blocks, and other localized pharmacologic agents and treatments. A port placement method utilizes ultrasound imaging for placement in proximity to patients' nerves and nerve centers. Neuromodulation procedures can also benefit from the ultrasound placement technology of the present invention.

### 2. Description of the Related Art

Various medical procedures involve the administration of pharmacologic agents for achieving favorable outcomes. For example, anesthesiology typically involves anesthetizing patients during surgery and other medical and dental procedures. General anesthetics render patients unconscious for limited, predetermined periods of time, during which medical procedures, e.g., surgeries, are performed. Local anesthetics are commonly used for anesthetizing specific areas of patients, e.g., for dental procedures, surgeries performed on extremities, etc.

Well-established local anesthetic injection procedures include nerve blocks, peripheral nerve blocks, epidural blocks, subarachnoid blocks and spinal blocks. The purpose of these blocks is to inhibit transmission of pain or sensation, thus terminating the pain signals received by the nervous system. These blocks can be used to treat acute pain, as is used for surgical procedures, as well as chronic pain, and have been shown to decrease opioid use. Other suitable medicines and drugs can be used to modulate or control pain, or extend the duration of nerve blocks, and are often added to local anesthetics or used alone. New medicines currently in development that work via specific sodium channels show promise in providing superior pain control.

The field of palliative care involves treating patients who have been diagnosed with serious illnesses. Palliative care objectives include improving patient quality of life and minimizing disruption for caregivers, e.g., medical professionals and family members. Palliative care is a growing field in medicine. Population demographics in the United States, including an aging population, are likely to contribute to more palliative care cases and greater anticipated demand for adequate pain relief. End-of-life patients are often treated by hospice care medical service providers. Patients receiving hospice care often require medications for chronic pain.

Opioids represent a significant class of pain control drugs and are commonly prescribed for and administered to patients dealing with chronic pain, including hospice care patients. However, opioid-based pharmaceuticals have multiple disadvantages. Patient addiction and opioid dependency are significant concerns. Expense and stringent regulatory (e.g., FDA) control are additional factors. Moreover, patients can develop tolerances, which can necessitate switching treatment protocols and increasing required dosages to achieve effective outcomes. Non-opioid options for pain control are desirable and needed due to the deleterious side effects of chronic opioid use. Chronic pain, palliative, and hospice patients often have increased pain control requirements requiring escalating doses of opioids. As doses increase, so do the side effects, often leading to consequences that decrease quality of life or lead to death of the patient.

Catheters for administering medications, including anesthetics via patients' venous circulatory systems, are well-known in the art. For example, Luther U.S. Pat. No. 5,403,283 discloses a percutaneous port catheter assembly and method of use. Cai et al. U.S. Pat. No. 5,743,873 discloses methods for using catheter connectors and portals, and methods of assembly.

Local anesthetics are generally most effective when administered in proximity to patients' nerves. Relatively recent improvements in ultrasound technology enable healthcare providers to more precisely visualize and locate nerves and nerve centers, as compared to blind catheter placement techniques used previously. Ultrasound technology has sufficiently advanced so that precise placement of injections near nerves or nerve centers can be performed with real-time ultrasound imaging machines at patients' bedsides. Such ultrasound imaging machines are generally superior to other current visualization modalities due to their size, portability, quality imaging, visualization of deep anatomic structures and an absence of ionizing radiation. Ultrasound imaging machines of this kind are termed point-of-care-ultrasound systems (POCUS). POCUS can remove the necessity of transporting patients for treatment which can be painful, expensive and deleterious to their physical conditions.

Ultrasound relies on reflection of sound waves generated by an ultrasound probe. The sound waves are recaptured and analyzed to generate live, real-time images. Ultrasound waves are reflected according to the target anatomic structures' physical properties, including density, fluid characteristics (e.g., viscosity and flow mechanics). Differences in these properties allow generation of the ultrasound images. These properties, however, are often similar to each other and to the materials used to construct catheter systems. Because of this, it is often difficult to discern catheters from adjacent anatomy. This can lead to complications such as nerve damage from needle trauma, inadvertent vein or artery puncture, and injection of local anesthetic into the vascular system which can produce a syndrome termed Local Anesthetic Systemic Toxicity (LAST), which can produce cardiovascular collapse and death. Certain devices and anatomic structures are discernable with ultrasound and are thus called echogenic.

Long-term, local anesthetic delivery is desirable in treating disorders such as complex regional pain syndrome (CRPS), peripheral neuropathy and postherpetic neuralgia, among many others. The delivery of new medicines, such as sodium channel specific local anesthetics, will be well-suited for such a catheter system. Currently available systems, called percutaneous catheters, describe catheters that transverse the skin layer. Percutaneous catheters allow a limited time period for treatment of four to six days before the catheter must be removed. After this time period, the risk of infection caused by a catheter passing from outside the body to inside the body can supersede the benefits of pain relief. The nerve block catheter-port system of the present invention addresses infection risk by adding a port below the skin (subcutaneous) for injecting medicines. The port attaches to the nerve block catheter. The entire system lies below the epidermis.

A uniquely echogenic catheter that is suited for long-term use would be an improvement to the art and is desirable and needed.

A nerve block is an injection to decrease inflammation or “turn off” a pain signal along a specific distribution of nerve or group of nerves. This is achieved by injecting numbing medicines, i.e., local anesthetics, and other pain-inhibiting drugs. There are over 40 nerve blocks that are used in medicine today. As ultrasound gets more advanced and the visualization of anatomy becomes more clear, additional blocks will be possible.

The purpose of peripheral nerve blocks is to inhibit impulse transmission in a nerve or group of nerves, thus terminating the pain signal perceived by the nervous system. Nerve blocks can be used to treat acute pain (e.g., during surgery), as well as for treatment of chronic pain. Impulse blockade can be brief (hours) or prolonged (days), depending on the medication used in the block and the technique. If short-term pain control (e.g., hours) is required, medication can be administered via single injections. Longer-term pain control (e.g., days) can be provided via a percutaneous catheter.

Nerve blocks have been shown to decrease the use of opioids because the sensation of pain from the site of surgery is greatly diminished or is absent. Single injection nerve blocks generally last for 12-24 hours and percutaneous nerve blocks can last for three to four days. Nerve blocks have the potential to decrease opioid use beyond this short time window when combined with the present invention to prolong their effectiveness.

Currently, catheters that are placed near a nerve are brought out through the skin (percutaneous) and attached to a pump that delivers a local anesthetic for approximately three to four days. This is very effective pain control but has limitations. The catheter travels from the pump, through the skin, to terminate near the nerve. Any foreign body (a catheter in this case) that passes through the skin can be an avenue for bacteria to make its way into the below the skin and cause infection. Research shows that the chance of bacterial infection rises each day. Hence it is not recommended for current percutaneous nerve block catheters to remain in place for more than four days.

Port-catheter systems have been used for decades in patients where access is needed to the venous system, i.e., the catheter is placed in a vein. A port-catheter system where the catheter is in a vein is used for patients who are receiving chemotherapy, need frequent blood transfusions, etc. The nerve block catheter-port system of the present invention provides a below-the-skin port in combination with a nerve block catheter. Although the port is similar, the nerve block catheter is very different than a catheter meant to be placed in a vein. For example, such catheters can be made of different materials, can be designed to be visualized with ultrasound, have different mechanical properties (stiffness, diameter), and have a different structure. Catheters intended for use in veins are generally not compatible with nerve block use.

### 3. Neuromodulation Background

Neuromodulation generally involves applying electrical impulses in the human body for therapeutic benefit. The modern era of neuromodulation began in the early 1960s with the use of deep brain stimulation (DBS) to resolve chronic and intractable pain, and evolved to include spinal cord stimulation by the end of the decade.

Neurosurgeon C. Norman Shealy has been credited with the first implantable neuromodulatory device for the relief of intractable pain in 1967. His spinal cord stimulators, which he called “dorsal column stimulators,” were intended exclusively for pain relief. These early efforts were not without complications, however, due in large part to mechanical shortcomings of the new devices.

By 1974, a group of physicians developed a less-invasive stimulating electrode. Implanting electrodes outside the subarachnoid space enabled stimulation to occur without side effects like spinal cord compression and leakage of cerebro-spinal fluid. Neurophysiologist Jan Holsheimer, PhD (University of Twente, The Netherlands), further optimized this work based on two decades of computer modeling research. His development of multiple electrode contacts has improved the understanding of the placement and design of electrical field stimulation onto spinal and brain targets. This has informed clinicians and manufacturers about how to better position the electrodes in the epidural space to enhance therapeutic benefits.

Neuromodulation therapy acts directly upon nerves. It generally involves altering or modulating nerve activity by delivering electrical or pharmaceutical agents directly to target areas. Neuromodulation devices and treatments can be life-changing. They can affect the entire body and treat a wide range of diseases and symptoms. Such conditions and treatments include headaches, tremors, urinary incontinence, deep brain stimulation (DBS) treatment for Parkinson's disease, sacral nerve stimulation for pelvic disorders and spinal cord stimulation for ischemic disorders (e.g., angina, peripheral vascular disease).

In addition, neuromodulation devices can stimulate a response where there was previously none, as in the case of a cochlear implant restoring hearing in a deaf patient. With such a broad therapeutic scope, and significant ongoing improvements in biotechnology, neuromodulation healthcare applications are expected to expand.

An emerging technology called the BrainGate Neural Interface System (https://www.braingate.org) has been used to analyze brain signals and translate those signals into cursor movements, allowing severely motor-impaired individuals an alternate “pathway” to control a computer with thought, and offers potential for one day restoring some degree of limb movement. Neuromodulation works by either actively stimulating nerves to produce a natural biological response, or by applying targeted pharmaceutical agents in tiny doses directly to site of action.

Neurostimulation devices involve the application of electrodes to the brain, the spinal cord or peripheral nerves. These precisely placed leads connect via an extension cable to a pulse generator and power source, which generates the necessary electrical stimulation. A low-voltage electrical current passes from the generator to the nerve, and can either inhibit pain signals or stimulate neural impulses where they were previously absent. In the case of pharmacological agents delivered through implanted pumps, the drug can be administered in smaller doses because it does not have to be metabolized and pass through the body before reaching the target area. Smaller doses—in the range of 1/300 of an oral dose—can mean fewer side effects, increased patient comfort and improved quality of life.

Heretofore there has not been available a pain management system and method with the advantages and features of the present invention. These advantages and features include, without limitation: ultrasound visualization for anatomic imaging for placing catheters near nerves and nerve centers (bundles); producing ultrasound images that are uniquely different from anatomic structures to enable accurately placing catheter infusion ports, which are the most crucial system components, near patients' nerves and nerve centers. The aforementioned complications can thus be minimized or avoided. Moreover, the ultrasound visualization procedures described herein can be used in connection with neuromodulation techniques.

### SUMMARY OF THE INVENTION

In the practice of an aspect of the present invention, a pain management system is provided that includes an implantable port with an elastomeric septum, which is connected to a catheter. The catheter can be placed with its infusion ports in proximity to nerves or nerve centers using ultrasound imaging techniques and equipment and is designed to be uniquely visible (echogenic) when viewed with ultrasound. The catheter system is located in a patient's body below the skin layer (subcutaneous) to minimize infection risk. The port provides a conduit for injecting medicines and can be readily felt from above the skin by medical professionals allowing for placement of the Huber needle into the port. The catheter is fluidically connected to the port with infusion ports at the distal end. A programmable microprocessor can be connected to a medication control system for dispensing predetermined medication quantities continuously or intermittently. Alternatively, a syringe can be used for manually introducing medication via the implantable port.

A nerve block system that is placed below the skin allows medicines to be administered long-term. After the skin above the port is sterilized, it is accessed by a specially designed needle (e.g., a Huber needle). After the nerve block has been given via either a one-time dose or an infusion, the Huber needle can be removed. If a long-term infusion is needed, the access needle needs to be removed and the skin re-sterilized, e.g., at weekly intervals. The nerve block catheter-port system of the present invention can be used to provide long-term pain control for palliative, hospice and chronic pain patients.

A medication delivery method according to the present invention includes the steps of placing an implantable port, extending a catheter from the port to an affected area requiring treatment, and injecting medication administration as necessary to achieve a favorable outcome, such as healing or alleviating pain and discomfort. Medications can be delivered intermittently or continuously.

In another embodiment, a MEMS (microelectromechanical system) placed in the catheter can monitor nerves or nerve centers for abnormal function and provide ultrasonic, electrical or physical treatment according to a pre-programmed algorithm. For example, if a nerve injury causes insolated nerve dysfunction, the nerve may not activate in a normal physiological pattern. The MEMS sensor is activated by this electrical dysfunction and treatment is provided via a pMUT (Piezoelectric micromachined ultrasonic transducers), neuromodulatory actuator or other device according to predetermined programming on the microchip included in the MEMS.

In another embodiment, a MEMS can monitor nerve or nerve center and relay information related to function and/or dysfunction. This information can be transmitted by direct or wireless electrical connection to a suitable computing machine and used to monitor or guide treatment.

In another embodiment, the vibratory qualities of the catheter tip could provide treatment. Vibratory therapy is known to inhibit nociceptive receptors and treat pain in the setting of peripheral neuropathy among other disorders and could be provided to very specific nerve or nerve centers by the oscillatory method provided by electromagnetic or electrical energy, the activation of a MEMS and other herein described embodiments.

In one embodiment the port is accessed intermittently or continuously with a Huber, or other non-coring needle. If used continuously, this needle can be removed periodically, the skin sterilized, and a new sterile needle introduced to minimize infection risk.

In another embodiment, an echogenic marker that is discernable from anatomy is placed at the tip of the catheter near the infusion ports or in the wall of the catheter as an aid in placement of the catheter system. The echogenic marker can be comprised of biocompatible materials such as ceramics, polymethyl methacrylate (PMMA), titanium, stainless steel, or other suitable material. This echogenic structure can provide the medical professional with confirmation that the infusion ports of the catheter system are located in the desired location in relation to nerves or nerve centers.

In another embodiment, a wire coil is embedded in the catheter creating a solenoid and electrical conductors are extended to the distal end of the catheter. An echogenic magnet is placed in the solenoid and the system is energized via a time-varying current producing oscillatory movement of the magnet as a unique ultrasonic visual aid in placement of the catheter system. The system is energized by a nerve stimulator, as is commonly available in the medical setting, or a similar device suitable to produce the magnitude of electromagnetic field necessary to produce oscillatory movement. Nerve stimulators supply power in a regular on/off pattern per a predetermined frequency. As the solenoid in the catheter is energized, the echogenic magnet is induced to move due to electromagnetic energy. When the solenoid is de-energized, the magnet returns to its original position. This oscillatory movement can provide a unique visual identifier to the medical professional, thus confirming that the infusion ports of the catheter system are located in the desired location in relation to nerves or nerve centers.

Electrorheological (ER) and magnetorheological (MR) fluids are special classes of materials that can respond to the electrical field and magnetic field, respectively, resulting in a physical change from a liquid to a solid. In another embodiment, an amount of ER fluid is placed in the catheter system and electrical conductors are extended to the distal end of the catheter and energized by a nerve stimulator or similar suitable device for energizing the fluid to induce a physical change thereby making the catheter system more discernable when viewed with ultrasound. Likewise, a physical change in a MR fluid could be induced by creating a magnetic field by the use of a solenoid as described in the previous embodiment.

Piezoelectric micromachined ultrasonic transducers (pMUTs) have recently been developed which are devices that are small enough to be placed in close physical location or direct contact with nerves or nerve centers. pMUTs are a version of a microelectromechanical system (MEMS) that incorporate a piezoelectric crystal for the purpose of converting electrical energy to sound energy and vice versa. In another embodiment, these or similar devices are installed at the distal end the catheter in the catheter system, allowing direct mechanical stimulation of nerve or nerve centers, allowing precise ultrasonic treatment. pMUTS can be energized via the aforementioned embodiments for energizing the catheter system.

In another embodiment, a phase change agent is provided in the catheter tip to facilitate ultrasound detection. In another embodiment, a stylet with a unique shape or device at its distal end could be placed in the catheter and used to induce movement of the distal end of the catheter to aid visualization with ultrasound.

### BRIEF DESCRIPTION OF THE DRAWINGS

FIG. 1 a cross-sectional view of a catheter system embodying an aspect of the present invention, shown receiving a medication for transfusion in a patient.

FIG. 2 is a flowchart showing a pain management treatment protocol method embodying an aspect of the present invention.

FIG. 3 is a fragmentary, cross-sectional view of an alternative embodiment catheter tip with an echogenic marker.

FIG. 4 is a fragmentary, cross-sectional view of another alternative embodiment catheter tip with a solenoid and magnet assembly.

FIG. 5 is a fragmentary, cross-sectional view of another alternative embodiment catheter tip with magnetorheological or electrorheological fluid.

FIG. 6 is a fragmentary, cross-sectional view of another alternative embodiment catheter tip with a phase change agent.

FIG. 7 is a fragmentary, cross-sectional view of another alternative embodiment catheter tip with a micro-electronic mechanical system or a piezo-electronic micro-machined ultrasonic transducer.

FIG. 8 is a fragmentary, cross-sectional view of another alternative embodiment catheter with a braided sleeve and an echogenic tip.

FIG. 9 a cross-sectional view of another alternative embodiment with a bent-end placement stylet configured for manipulation to facilitate ultrasound visibility and accurate placement, shown being positioned in a patient.

FIG. 10 is an enlarged, fragmentary view of the bent-end stylet.

FIG. 11 is a cross-sectional view of yet another alternative embodiment with a neuromodulation subsystem configured for wirelessly transmitting variable frequency signals from a transmitting coil to a subcutaneous receiving coil for neuromodulation via a catheter.

FIG. 12 is cross-sectional view of another alternative embodiment catheter with multiple coil segments for enhanced visualization and neuromodulation.

FIG. 13 is a flowchart showing a pain management and neuromodulation treatment method embodying an aspect of the present invention.

FIG. 14 is a cross-sectional view of another alternative embodiment catheter system with neuromodulation embodying an aspect of the present invention, shown with an inlet port embedded in a patient and receiving an injected quantity of medication for administration to a patient.

FIG. 15 is a cross-sectional view thereof, particularly showing a stylet configured for placing the catheter, which can include wire stimulation leads for neuromodulation. The stylet can include a bent-wire end for twirling to facilitate observation using ultrasound.

FIG. 16 is an enlarged, perspective view of a detachable catheter tip configured for guiding the stylet into the catheter.

FIG. 17 is an exploded view of an implantable inlet port, showing a barb for fluidic connection to the catheter.

FIG. 18 is a fragmentary, side-elevational view of a catheter including an internal braid and a stimulation tip.

### DETAILED DESCRIPTION OF THE PREFERRED EMBODIMENTS

### I. Introduction and Environment

As required, detailed aspects of the present invention are disclosed herein, however, it is to be understood that the disclosed aspects are merely exemplary of the invention, which may be embodied in various forms. Therefore, specific structural and functional details disclosed herein are not to be interpreted as limiting, but merely as a basis for the claims and as a representative basis for teaching one skilled in the art how to variously employ the present invention in virtually any appropriately detailed structure.

Certain terminology will be used in the following description for convenience in reference only and will not be limiting. For example, up, down, front, back, right and left refer to the invention as orientated in the view being referred to. The words, “inwardly” and “outwardly” refer to directions toward and away from, respectively, the geometric center of the aspect being described and designated parts thereof. Said terminology will include the words specifically mentioned, derivatives thereof and words of similar meaning. The definition of nerve designates peripheral nerves such as are commonly known, such as the saphenous, femoral, intercostal or radial nerves as well as numerous others. Nerve centers designate more proximal (e.g., closer to the central nervous system) nerve locations than peripheral nerves and include, but are not limited to, nerve roots, trunks, divisions, cords, and plexuses. Examples of these are the cervical roots, anterior division, lateral cord and the brachial plexus. Likewise, epidural or subarachnoid, as they are commonly referred to in the art, are an anatomic location that contains nerves, nerve roots and/or plexuses.

### II. Ultrasound-Guided Catheter System 2

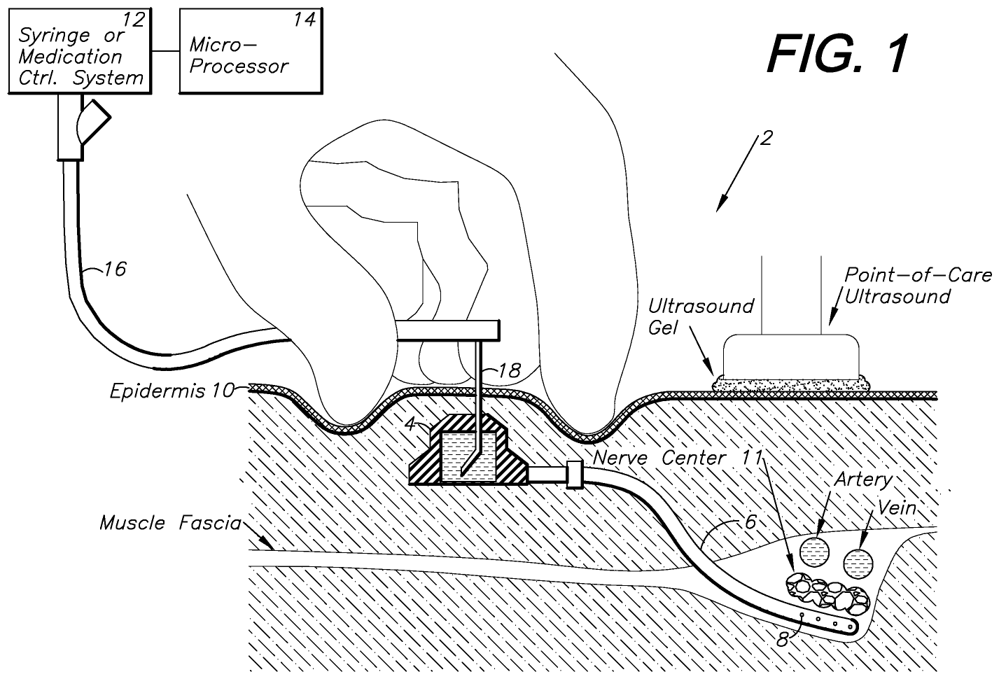

A catheter system 2 embodying an aspect of the present invention is shown in FIG. 1 and generally includes an implantable port 4 connected to a catheter 6. The port 4 is preferably placed below the epidermis 10, and can be internally attached with sutures, surgical staples or some other attachment mechanism. The port 4 is preferably configured for receiving a quantity of a pharmacologic treatment, such as an anesthetic, a chemotherapy medication, etc. The port 4 can be constructed of ferrous or non-ferrous materials with a reservoir 5 and is designed for coupling with the catheter 6 via a coupler 7. The coupler 7 can be designed for repeated detachment from and attachment to the catheter 6 to facilitate replacement of the port 4 and catheter 6.

Additionally, it may be desired to temporarily detach the port 4 from the catheter 6 to inject pharmacologic agents into the catheter 6 to evaluate the placement of those medicines near nerves or nerve centers or evaluate function of the catheter 6. It may be desirable for the port 4 to be of smaller size in its height and diameter to allow for placement in a variety of anatomic locations to minimize discomfort and decrease the likelihood of skin ulceration due to pressure from outside the body to the skin over the port. The catheter 6 is fluidically connected to the port 4 and terminates in proximity to portions of the patient's nervous system to be anesthetized. Nerves and nerve centers 11 can be located using an ultrasound imaging procedure.

For placement of the catheter 6, a Tuohy needle, or some other suitable non-coring needle, is advanced under ultrasound guidance to a nerve or nerve center 11. The catheter is then advanced through the needle and the needle is withdrawn. In another embodiment, known as the Seldinger technique, a needle is advanced to the nerve or nerve center 11, a wire is advanced through the needle, the needle is withdrawn, and the catheter 6 is exchanged over the wire.

In another embodiment commonly used for catheter placement, a breakaway sheath and wire system accomplishes the same purpose.

The catheter 6 of the system 2 is located so that infusion ports 8 of the catheter 6 are near a nerve or nerve center 11, but the port portion of the system is located in an anatomic location that is convenient for access of injection as well as comfort of the patient. A path below the skin from the catheter to the port can be created by tunneling, as is well known in the art for the placement of ventriculoperitoneal shunts or spinal cord stimulators, if the desired location for the port is some distance from the catheter 6.

As an example, it may be necessary, as in the case of epidural placement of the catheter 6 in the midline of the back, for the port to be located on the patient's flank to prevent ulceration of the skin covering the port, for comfort of the patient and ease of injection. The catheter 6 can be trimmed to length so the distance from the infusion ports 8 of the catheter 6 to the port can be specifically tailored for each patient, allowing the port to be placed in an anatomic location convenient for injection and with adequate fascia layers for securing the port with sutures or another method, with its septum facing outward.

A syringe or other medication control system 12 is connected to tubing 16 terminating at a needle 18. Huber and other suitable non-coring needles can be used for injecting medications into the subcutaneous port 4. The needle 18 can also comprise a stylet, which can be curved. Various medication dispensing devices can be used with the catheter system 2 of the present invention. For example, in lieu of a manually-operated syringe, a motorized pump can be provided. Moreover, operation of the medication control system 12 can be automated with a programmable microprocessor 14 for cycling the operation of a motorized pump to dispense medication at predetermined intervals consistent with a predetermined treatment protocol. Medicine control systems such as described are currently available and commonly used in medical settings. Moreover, various medications can be selectively administered, including, without limitation, anesthetics, chemotherapy medications, growth factors, antibiotics, etc.

The port 4 can be accessed intermittently or continuously with a Huber or other non-coring needle 18. If used continuously, this needle 18 can be removed periodically, the skin 10 sterilized, and a new sterile needle 18 introduced to minimize infection risk.

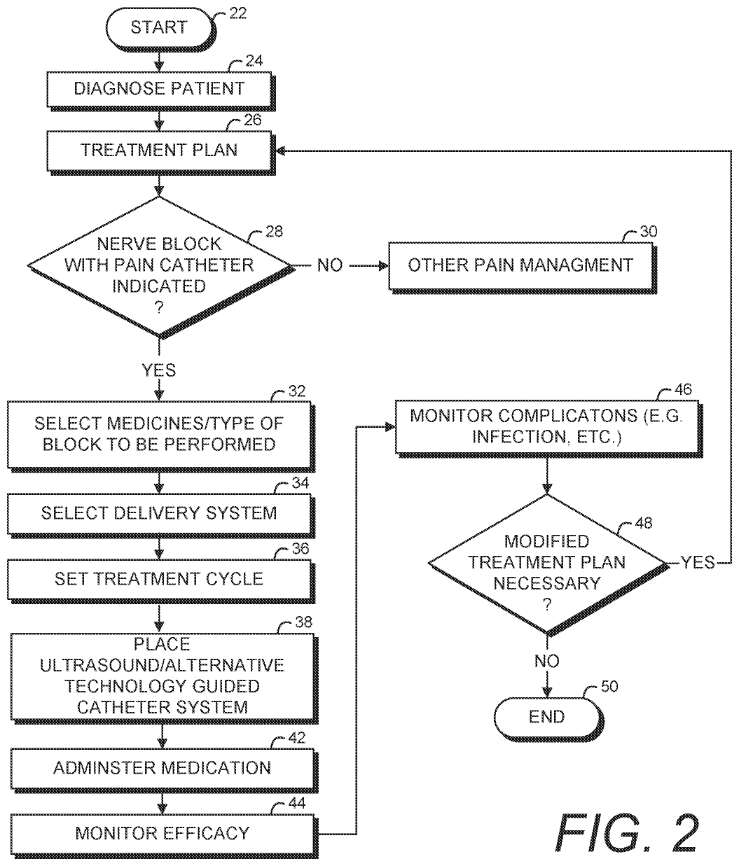

FIG. 2 is a flowchart showing an example of a medication administration method embodying an aspect of the present invention. From a start 22, the method includes the steps of diagnosing the patient at 24 and devising a treatment plan at 26. At decision box 28, a procedure including a nerve block with a pain catheter can be considered. If negative (“NO”), other pain management can be chosen at 30. If positive (“YES”), the protocol continues to selection of medicines and type of block to be performed at 32 and selection of a delivery mechanism at 34. The treatment cycle (e.g., intermittent, continuous, etc.) is set at 36. Step 38 involves placing an ultrasound/alternative technology guided catheter system.

Medication is administered at 42 and its efficacy is monitored at 44. Complications are monitored at 46 and can include, for example, infection, toxicity, etc. If a modified treatment plan is deemed necessary (“Yes” path from decision box 48), the protocol loops back to the treatment plan step 26. If “No,” the treatment terminates at 50.

III. First Alternative Embodiment Catheter System 102 with Echogenic Marker 104

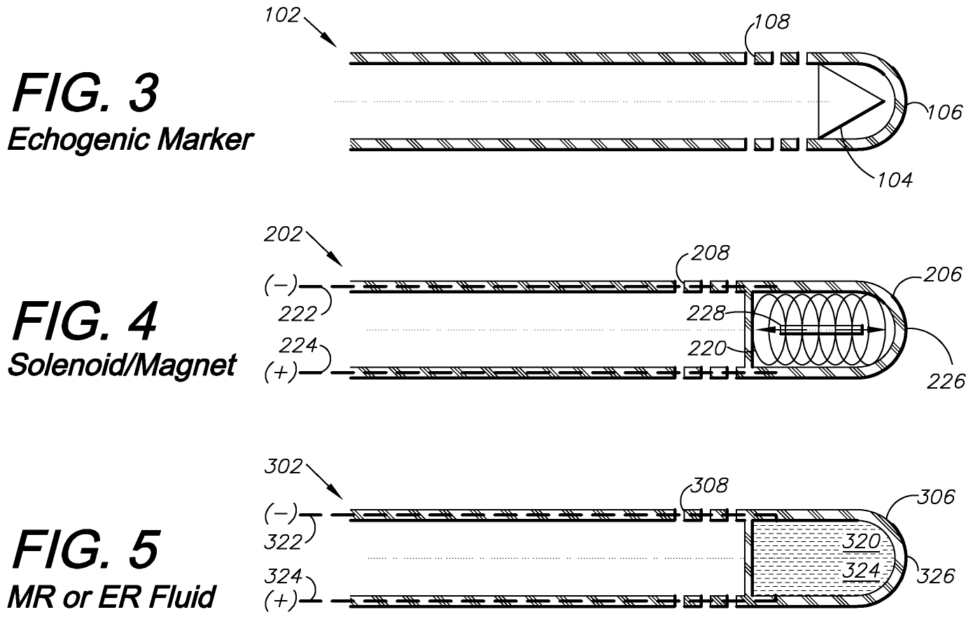

In another embodiment or aspect of the present invention, shown in FIG. 3, an echogenic marker 104 that is discernable from anatomy is placed at the tip of a catheter 106 near infusion ports 108 in the wall of the catheter 106 as an aid in placement of the catheter system 102. The echogenic marker 104 can be comprised of biocompatible materials such as ceramics, polymethyl methacrylate (PMMA), titanium, stainless steel, or other suitable material. This echogenic structure can provide the medical professional with confirmation that the infusion ports 108 of the catheter system 102 are located in the desired location in relation to nerves or nerve centers 11.

IV. Second Alternative Embodiment Catheter System 202 with Solenoid

In another alternative embodiment or aspect of the present invention, shown in FIG. 4, a wire coil solenoid 220 is embedded in a catheter 206. Electrical conductors 222 (−) and 224 (+) are extended to a distal end 226 of the catheter 206. An echogenic magnet 228 is placed in the solenoid 220 and the system 202 is energized via a time-varying current producing oscillatory movement of the magnet 228 as a unique ultrasonic visual aid in placement of the catheter system 202. The system is energized by a nerve stimulator, as is commonly available in the medical setting, or a similar device suitable to produce the magnitude of electromagnetic field necessary to produce oscillatory movement.

Nerve stimulators can supply power in a regular on/off pattern per a predetermined frequency. As the solenoid 220 in the catheter is energized, the echogenic magnet 228 is induced to move due to electromagnetic energy. When the solenoid is de-energized, the magnet returns to its original position. This oscillatory movement can provide a unique visual identifier to the medical professional, thus confirming that infusion ports 208 of the catheter system 202 are located in the desired location in relation to nerves or nerve centers 11.

V. Third Alternative Embodiment Catheter System 302 with Electrorheological (ER) and Magnetorheological (MR) Fluids

In a third alternative embodiment or aspect of the invention shown in FIG. 5, a catheter system 302 includes a quantity of ER or MR fluid 320 placed in a reservoir 321 in the catheter distal end 326. The catheter system 302 includes infusion ports 308. Negative and positive electrical conductors 322, 324 are extended to the catheter distal end 326 and energized by a nerve stimulator or similar suitable device for energizing the fluid 320 to induce a physical change, thereby making the catheter 306 more discernable when viewed with ultrasound. Likewise, a physical change in a MR fluid could be induced by creating a magnetic field by the use of a solenoid as described in the previous embodiment 202.

VI. Fourth Alternative Embodiment Catheter System 402 with Phase Change Agent

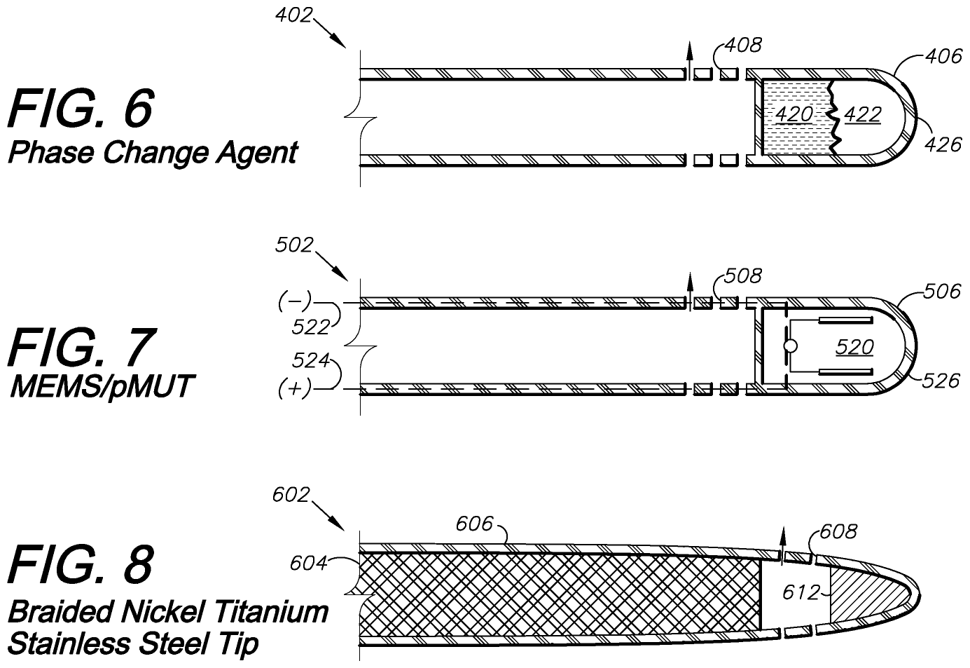

FIG. 6 shows a catheter system 402 comprising another alternative embodiment or aspect of the present invention with a phase change agent 420 contained within a reservoir 422 in a distal end 426 of a catheter 406 with infusion ports 408. Phase change agents, such as, but not limited to perfluorocarbons, have a boiling point near body temperature so that vaporization can be induced by the acoustic energy provided by the ultrasound probe producing expansion in volume. This volume change is visible via ultrasound. In another embodiment, phase change agents are encapsulated in the catheter system and induced by energy of the ultrasound probe to create a unique visual marker. When the ultrasound probe is removed, the phase change agent returns to the prior state.

VII. Fifth Alternative Embodiment Catheter System 502 with MEMS or pMUT

FIG. 7 shows a catheter system 502 embodying a fifth alternative embodiment or aspect of the present invention with a microelectromechanical system (MEMS) or a piezoelectric micro-machined ultrasonic transducer (pMUT) component 520. The component 520 is located in a closed, distal end 526 of a catheter 506 with infusion ports 508. Electrical conductors 522 (−) and 524 (+) are connected to a nerve stimulator, which can sequentially energize and deenergize the component 520 to achieve a desired result by varying the amplitude and frequency of the energizing signals.

The Microlectromechanical System (MEMS) catheter system 502 can be fabricated using semiconductor materials and incorporating mechanical components, sensors, actuators, and electronic elements with feature sizes ranging from a few millimeters to microns gauge. In another embodiment, MEMS can be incorporated in the catheter system and energized to induce a movement of a portion of the catheter system via an actuator that would be uniquely visible via ultrasound.

A distinct advantage of the herein described catheter system 502 is that the location of the port is immediately below the skin layer, minimizing distance to the external energy source, with the result of maximizing transfer efficiencies. The underside of the port, opposite of the septum, is geometrically suitable for placement of a receiving coil to be in parallel with a transmitting coil placed outside the skin. The coil that is embedded on the port can be connected to a conducting lead which is embedded in the catheter and terminating at the distal of end of the catheter in proximity to nerves or nerve centers and designed to conduct neuromodulation signals.

In another embodiment, ultrasonic energy transfer can be utilized to transcutaneously energy the catheter system. This utilizes the known piezoelectric effect that utilizes the conversion of ultrasonic energy to electrical energy. Ultrasonic transfer of energy allows longer power transmission distances and is free of electromagnetic interference. In this scheme, a piezoelectric transducer that is external to the skin layer faces a piezoelectric receiver embedded in the port and under the skin layer allowing transmission ultrasonic energy in either direction without penetrating the skin layer. Energy is transmitted through the skin layer via ultrasonic energy from the transducer to the receiver and is converted to electricity. Electrical energy can then be utilized to energize the other herein described embodiments.

VIII. Sixth Alternative Embodiment Catheter System 602 with Catheter Liner 604

FIG. 8 shows another embodiment catheter system 602 with a catheter liner 604 received in a catheter 606 including infusion ports 608. The liner 604 can comprise braided nickel titanium with a construction similar to medical, intravascular stents. A stainless-steel tip 612 is provided for echogenic visibility to facilitate placement.

IX. Seventh Alternative Embodiment Catheter System 702 with Stylet Locator 710

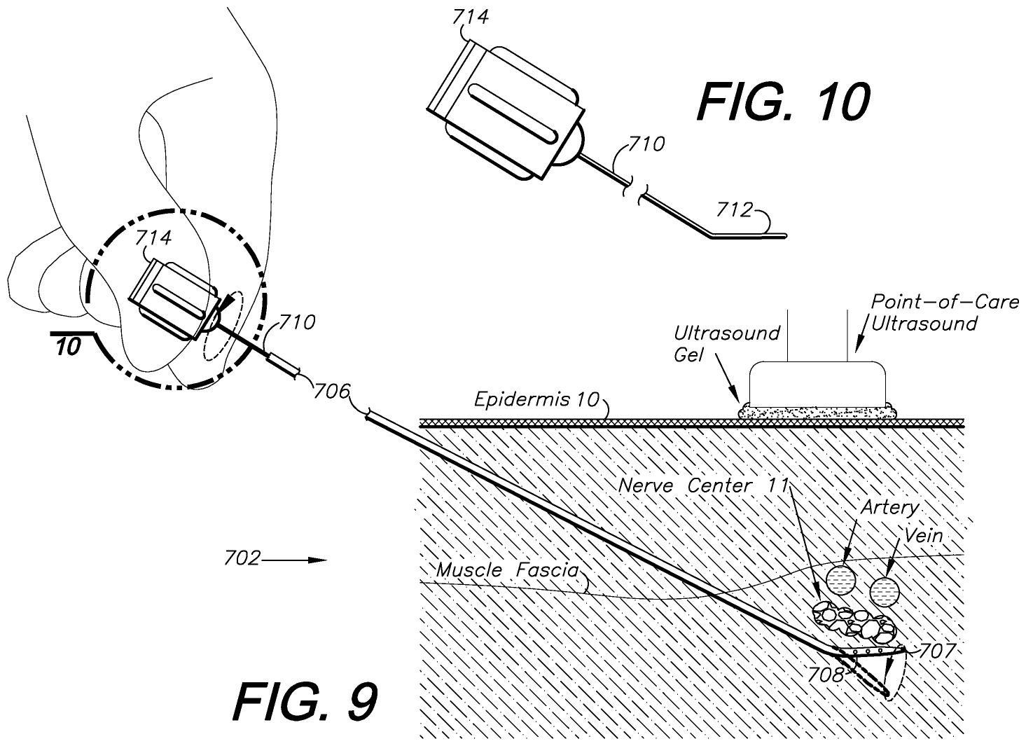

FIGS. 9 and 10 show another embodiment catheter system with a catheter 706 including a catheter end 707 and infusion ports 708. The catheter 706 can be configured for receiving a stylet 710, provided the passage of the catheter 706 has a sufficient internal diameter (ID). The stylet 710 includes a bent distal end 712 and a proximal end knob 714. As shown in FIG. 9, manually twirling the knob 714 causes a corresponding rotation of the stylet bent distal end 712, which facilitates ultrasound visibility for monitoring and accurate placement. In other words, a physician's “signature” manual manipulation of the stylet-catheter combination facilitates placement in proximity to a nerve center 11 for optimizing treatment effectiveness and patient outcomes.

X. Eighth Alternative Embodiment Catheter System 802 with Stylet Locator and Neuromodulation Pain Management

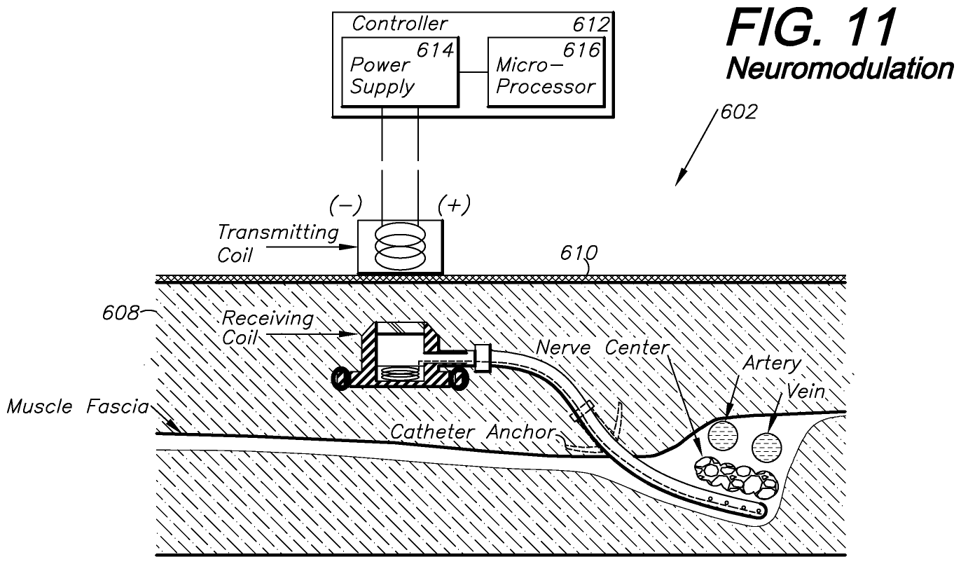

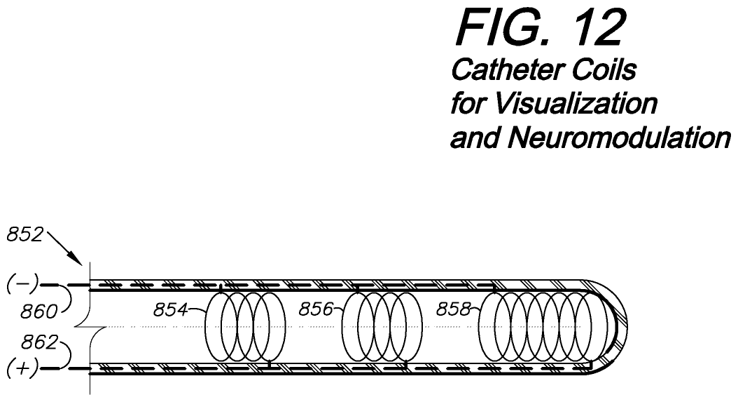

FIG. 11 shows a pain management system 802 with a neuromodulation subsystem 804, including a controller 812 with a power supply 814 and a microprocessor 816. A transmitting coil 818 is connected to the power supply 814 and is configured for placement on the patient epidermis 10. A receiving coil 820 is connected to a catheter 822, which can be embedded in the dermis 808 and secured by a catheter anchor 822. The receiving coil 820 is configured for receiving medication for placement via the catheter 11 as well as neuromodulation signal transmissions via the transmitting coil 818.

XI. Ninth Alternative Embodiment Catheter System 852 with Stylet Locator and Neuromodulation Pain Management

FIG. 12 shows another alternative embodiment catheter 852 with proximal, intermediary and distal coils 854, 856 and 858, respectively, placed internally in the catheter and connected to electrical leads 860, 862.

XII. Ultrasound-Placed Pain Management Methods with Subcutaneous Catheters and Neuromodulation

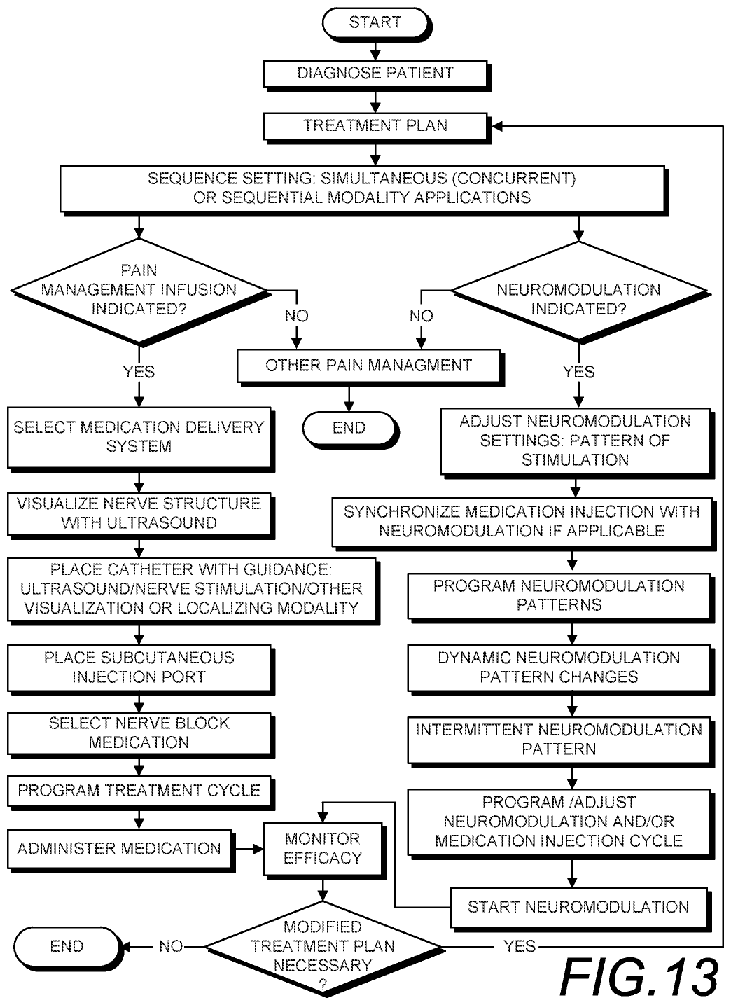

FIG. 13 is a flowchart showing the steps of applying the ultrasound-placed pain management and neuromodulation system according to the method of the present invention. As shown, pain management medication and the neuromodulation treatment methods can be used alternatively or in combination for achieving optimum patient outcomes. The ultrasound-placed pain management systems with subcutaneous catheters can be utilized for a variety of treatment protocols. Moreover, they are adaptable for a variety of medications. Automated systems, e.g., with programmable microprocessors, can be programmed for providing consistent, regular treatments as indicated. Moreover, patients' healing progress can be closely monitored and treatment protocols adjusted or terminated for achieving optimal patient outcomes.

### XIII. Tenth Alternative Embodiment Catheter System 902

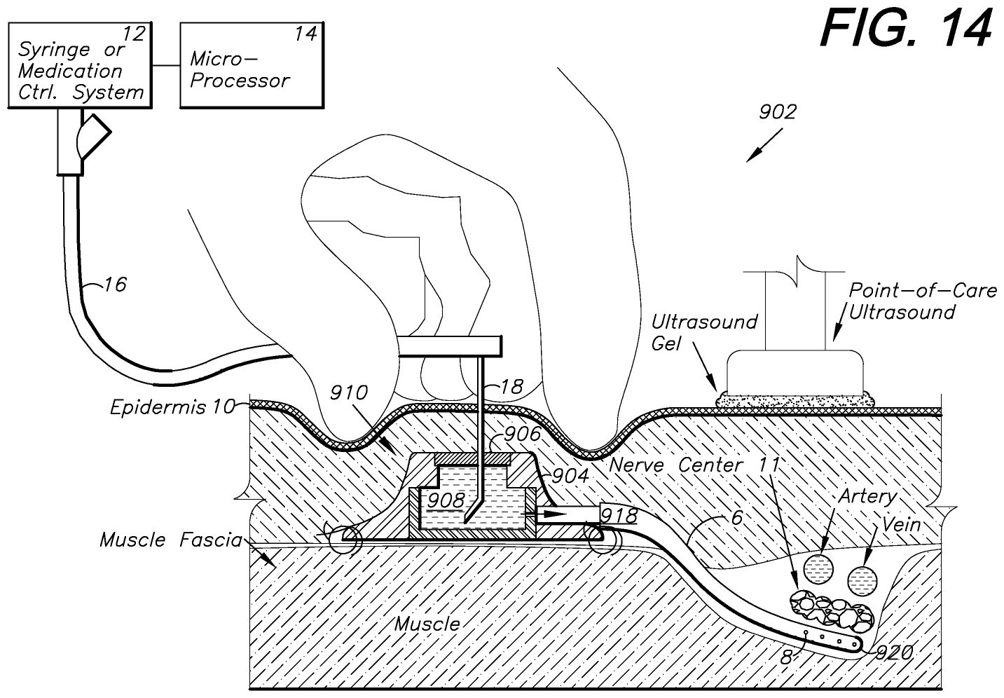

FIGS. 14-18 show a tenth alternative embodiment pain management system 902 including a subcutaneous port 904 connected to a catheter 6 including outlet ports 8. As with the previously described embodiments, the catheter system 902 is configured for delivering pain medication in close proximity to a patient's nerve center 11.

The port 904 includes a septum 906 enclosing a medication reservoir 908 configured for receiving medication injections via a needle 18 connected to a syringe or medication control system 12 via tubing 16. Optionally, a programmable microprocessor 14 can be connected to the syringe or medication control system 12 for selectively controlling medication injection parameters, including quantities, pressures, durations, timing, etc.

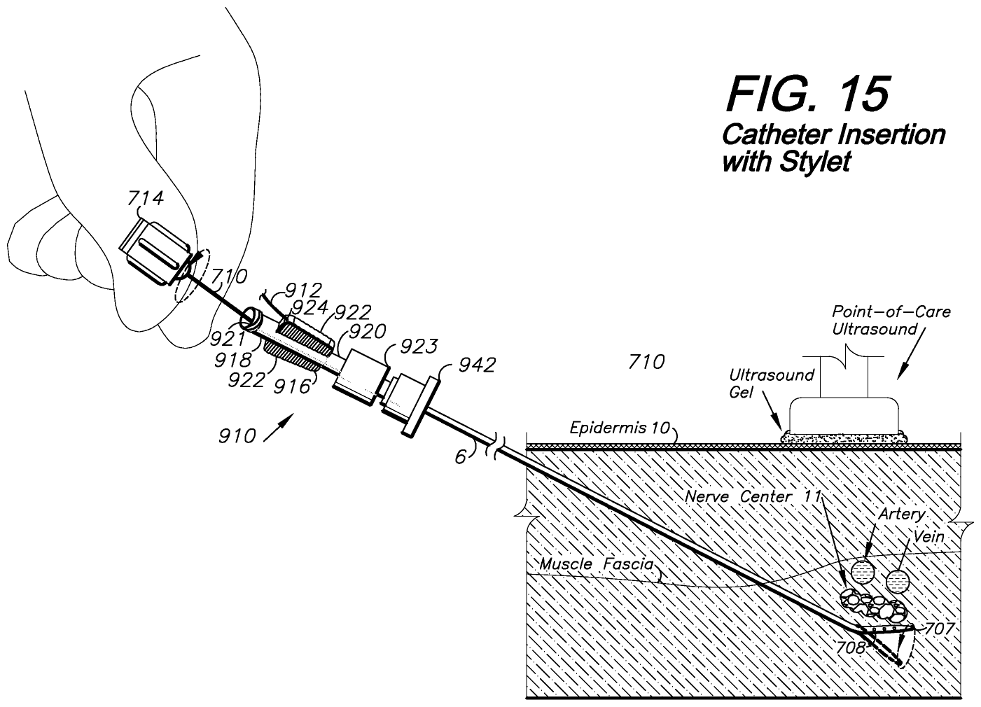

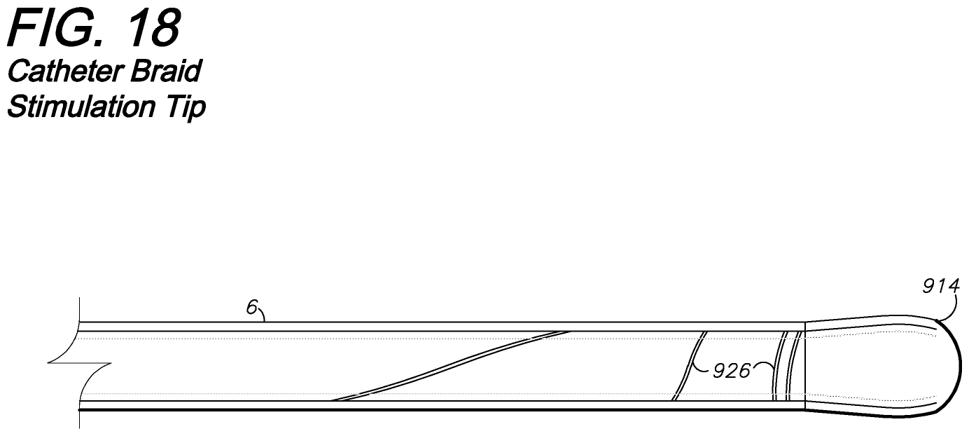

FIG. 15 shows a modified catheter hub assembly 910 configured for guiding placement of the catheter 6, which can include stimulation wire leads 912 extending to a catheter stimulation tip 914 as shown in FIG. 18. The catheter hub assembly 910 includes a catheter hub 916 with a proximal end 918 configured for receiving a stylet 710 for catheter placement as described above and a distal end 920. The catheter hub distal end 920 mounts a detachable tip 923 (FIG. 15) enabling the catheter 6 to be re-flushed after removing the catheter hub 916. A catheter proximal fitting 942 can be attached to the hub distal end 920 and mounts a proximal end of the catheter 6.

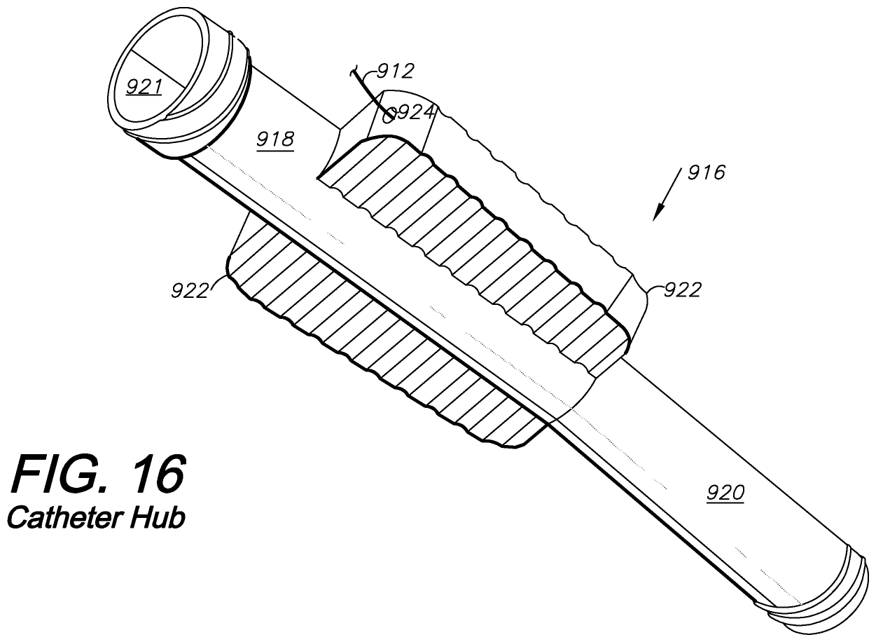

As shown in FIG. 16, the catheter hub 916 proximal and distal ends 918, 920 are slightly angled off-axis with respect to each other. An angled cavity 921 extends between and is open at the hub ends 916, 918. The angled configuration of the cavity 921 facilitates insertion of the angled stylet 710 into the catheter 6. Lateral flanges 922 extend laterally from the catheter hub 916 proximal end 918. One or both of the lateral flanges 922 can include a wire lead passage 924 configured for guiding a stimulation wire lead 912 into the catheter 6, e.g., for connection to the catheter stimulation tip 914.

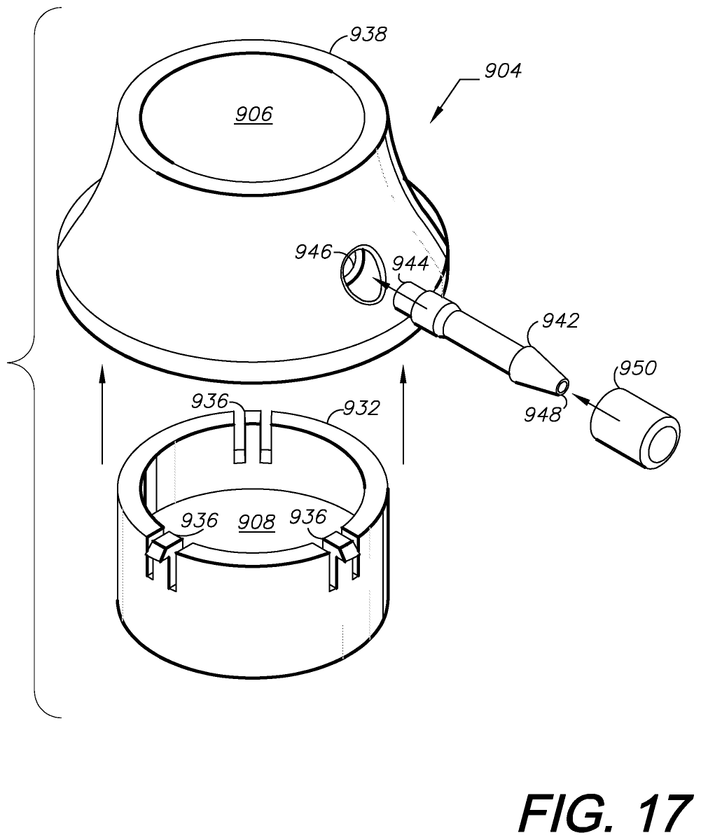

FIG. 17 is an exploded view of the subcutaneous port 904, which includes a base 932 forming the medication reservoir 908 and radially-spaced snap hooks 936 configured for snapping the base 932 into a port cover 938. A catheter proximal fitting 942 includes an inner end 944 configured for insertion into a side passage 946 of the port base 932 for discharging medication from the medication receiver 908 via an outer end 948 of the catheter proximal fitting 942. A sleeve 950 is configured for placement over the catheter proximal fitting outer end 948.

As shown in FIG. 18, a polymeric catheter braid 926 can be provided within the catheter 6 for flexibility and kink resistance. The braid 926 can comprise other materials, including metals. For example, NiTi-based alloys exhibit super elastic behavior, reversible strains during heating or cooling, low stiffness, nearly bone-comparable mechanical behavior and excellent corrosion resistance. Such properties favorably accommodate a variety of medical, dental and orthodontic applications.

The catheter systems and methods of the present invention can be adapted to accommodate a variety of medical conditions and treatment protocols. For example, and without limitation, antibiotics for infection control and growth factors for promoting re-epithelialization can be introduced to a wound site.

It is to be understood that while certain embodiments and/or aspects of the invention have been shown and described, the invention is not limited thereto and encompasses various other embodiments and aspects.

Having thus described the invention, what is claimed as new and desired to be secured by Letters Patent is:

1. A catheter system for pain management, which system includes:
a subcutaneous port including a medication delivery subsystem with a medication reservoir formed in said subcutaneous port;
a catheter fluidically connected to said medication reservoir and including a discharge port configured for discharging said medication in proximity to a patient's nerve center;
a medication dispenser configured for placement externally to the patient and including a hypodermic needle;
said medication port configured for receiving medication from said dispenser;
an echogenic catheter configured for fluidic connection to said medication port and terminating at a distal end internally to the patient;
a point-of-care ultrasound imaging system configured for monitoring placement of said echogenic catheter;
a medication control system connected to said medication dispenser and configured for selectively injecting medication to said catheter;
said medication control system configured for injecting medication in predetermined dosages;
said point-of-care ultrasound imaging system configured for imaging said catheter and patient nerve or nerve centers in real time;
said point-of-care ultrasound imaging system configured for use in guiding said catheter for medication discharge in proximity to a patient nerve or nerve center;
a stylet including a proximal end mounting a knob and a distal end; and
said stylet configured for insertion into said catheter and for catheter placement in a desired location within the patient.

2. The catheter system according to claim 1, which includes:
said stylet having a proximal section extending from said knob and a distal section extending from said proximal section and terminating at said distal end; and
said proximal and distal sections forming a bent, angular orientation relative to each other.

3. The catheter system according to claim 2, wherein said stylet distal section within said catheter is configured for manipulation via said knob to create an interactive signature visible with said ultrasound imaging system, said signature facilitating catheter distal end placement in proximity to a patient's neurology in real time.

4. The catheter system according to claim 3 wherein said catheter includes:
a catheter proximal end;
a catheter distal end with said discharge port; and
a catheter hub assembly mounted on said catheter proximal end and configured for fluidically connecting said catheter to said medication dispenser.

5. The catheter system according to claim 4, wherein said catheter hub assembly includes:
a catheter hub with proximal and distal ends;
said catheter hub proximal end configured for fluidic connection to said subcutaneous port;
said catheter distal end connected to said catheter; and
a pair of lateral flanges mounted on and extending laterally from said catheter hub proximal end.

6. The catheter system according to claim 5, which includes:
a catheter proximal fitting connected to said catheter proximal end;
a detachable tip connecting said catheter hub distal end to said catheter proximal fitting; and
said detachable tip configured for enabling catheter flushing after disconnection from said catheter proximal fitting.

7. The catheter system according to claim 6, which includes:
a metallic stimulation tip formed in said catheter distal end and configured for stimulating a patient's neurology;
a stimulation wire lead extending from a location external to the patient to said stimulation tip; and
an electrical pulse generator configured for placement external to the patient and connected to said stimulation wire lead.

8. The catheter system according to claim 7, wherein each said catheter hub lateral flange includes a stimulation lead passage configured for receiving said stimulation wire lead.

9. The catheter system according to claim 8, which includes:
a catheter braid with a spiral configuration and located within said catheter;
said catheter braid extending from said catheter proximal end to said catheter distal end; and
and said catheter braid comprising one of a polymeric material and a nickel titanium (NiTi) material.

10. The catheter system according to claim 1, which is configured for administering nerve block or other medications for treating nerve-related conditions.

11. The catheter system according to claim 1, which includes:
subcutaneous port retainers comprising sutures; and
a catheter anchor including prongs configured for embedding in subcutaneous tissue.

12. The catheter system according to claim 1, which includes:
said medication delivery subsystem including a medication reservoir formed in said subcutaneous port and configured for receiving an injection of medication; and
said catheter fluidically connected to said medication reservoir and including a discharge port configured for discharging said medication in proximity to the nerve center.

13. The catheter system according to claim 1, which includes:
a control subsystem with a microprocessor and a medication dispensing pump connected to said microprocessor and said catheter proximal end; and
said microprocessor configured for programming for selectively activating said medication dispensing pump.

14. The catheter system according to claim 13, which includes:
said medication dispensing pump configured for placement externally to the patient and including a hypodermic needle;
said medication reservoir configured for receiving medication from said dispenser;
said medication port configured for placement subcutaneously to the patient and fluidic connection to said medication dispensing pump;
an echogenic catheter configured for fluidic connection to said medication port and terminating at a distal end internally to the patient; and
a point-of-care ultrasound imaging system configured for visualizing and monitoring placement of said echogenic catheter.

15. The catheter system according to claim 6, which includes:
said catheter configured for ultrasound visualization and terminating in proximity to a nerve center of the patient.

16. The catheter system according to claim 7, which includes:
a medication control system connected to said medication dispenser and configured for selectively injecting medication to said catheter.

17. A catheter system for pain management, which system includes:
a subcutaneous port including a medication delivery subsystem with a medication reservoir formed in said subcutaneous port;
a catheter fluidically connected to said medication reservoir and including a discharge port configured for discharging said medication in proximity to a patient's nerve center;
a medication dispenser configured for placement externally to the patient and including a hypodermic needle;
said medication port configured for receiving medication from said dispenser;
an echogenic catheter configured for fluidic connection to said medication port and terminating at a distal end internally to the patient;
a point-of-care ultrasound imaging system configured for monitoring placement of said echogenic catheter;
a medication control system connected to said medication dispenser and configured for selectively injecting medication to said catheter;
said medication control system configured for injecting medication in predetermined dosages;
said point-of-care ultrasound imaging system configured for imaging said catheter and patient nerve or nerve centers in real time;
said point-of-care ultrasound imaging system configured for use in guiding said catheter for medication discharge in proximity to a patient nerve or nerve center;
a stylet including a proximal end mounting a knob and a distal end;
said stylet configured for insertion into said catheter and catheter placement in a desired location within the patient;
said subcutaneous port including a base with said medication reservoir and multiple snap hooks;
said subcutaneous port further including a port cover configured for snapping over said subcutaneous port base with said snap hooks;
said port cover including a septum configured for penetration by a hypodermic needle for introducing a quantity of medication into said base education reservoir;
said subcutaneous port cover including a side passage fluidically connected to said medication reservoir; and
said subcutaneous port cover including a catheter proximal fitting with a proximal end inserted in said side passage and a barbed distal end configured for inserting in said catheter proximal end and selectively receiving a closure sleeve configured for closing said catheter proximal fitting.

18. A method of managing pain, which method includes the steps of:
placing a medication dispenser externally to the patient;
placing a medication port subcutaneously to the patient;
injecting medication into said port;
connecting said medication dispenser and said port with tubing terminating at a needle;
placing a catheter subcutaneously;
connecting said catheter to said port;
terminating said catheter at a distal end in proximity to a patient nerve center;
discharging medication internally within the patient via the catheter;
providing a programmable controller for controlling medication discharge;
programming said controller for discharging medication in predetermined dosages to said port;
discharging medication at predetermined time intervals;
providing a point-of-care ultrasound imaging system configured for placing said catheter;
imaging patient neurology with a point-of-care ultrasound imaging system; and
imaging said catheter and patient nerve centers in real time with said point-of-care ultrasound imaging system;
guiding said catheter for medication discharge in proximity to a patient nerve center with said point-of-care ultrasound imaging system; and
administering nerve block medications and other medications for treating nerve-related diseases.

19. The method according to claim 18, which includes the additional steps of:
providing an echogenic marker located in said catheter distal end;
providing a solenoid coil located in said distal end of said catheter;
said solenoid coil configured for connection to an electrical power source via electrical leads within said catheter; and
providing an echogenic magnet reciprocably received in said solenoid coil and configured for ultrasonic detection and monitoring.

20. The method according to claim 18, which includes the additional steps of:
providing a quantity of electrorheological fluid encapsulated in said catheter distal end;
providing a quantity of magnetorheological fluid encapsulated in said catheter distal end;
providing a quantity of a phase change agent encapsulated in said catheter distal end; and
providing a microelectro-mechanical system encapsulated in said catheter distal end.
